(19) United States
(12) Patent Application Publication
(10) Pub. No.: US 2009/0285789 A1
(43) Pub. Date: Nov. 19, 2009

(54) METHODS FOR ENHANCING INNATE AND ADAPTIVE IMMUNITY AND ANTIGEN IMMUNOGENICITY

(75) Inventors: Ching-Liang Chu, Miaoli County (TW); Tzu-Chih Chen, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: YEASTERN BIOTECH CO., LTD, Taipei (TW)

(21) Appl. No.: 12/467,126

(22) Filed: May 15, 2009

Related U.S. Application Data
(60) Provisional application No. 61/054,081, filed on May 16, 2008.

(51) Int. Cl. A61K 36/074; A61P 37/00

(52) U.S. Cl. 424/93.21; 424/195.15

(57) ABSTRACT

The present invention is related to a method for enhancing innate and adaptive immunity by activating dendritic cells (DCs) and macrophages, comprising administering a subject LZ-8 protein. The present invention is also related to a composition for enhancing innate and adaptive immunity, comprising LZ-8 protein.

### CROSS-REFERENCE TO RELATED APPLICATIONS

The present invention claims priority to U.S. Application No. 61/054,081, filed May 16, 2008, which is incorporated herein by reference in its entirety.

### FIELD OF THE INVENTION

This invention relates to the method of applying LZ-8 (or FIP-gts), a fungal immunomodulatory protein (FIP) isolated from Ganoderma lucidum (G. lucidum, or G. tsugae), to promote the maturation of dendritic cells (DCs). It also relates to the method of using LZ-8 to induce pro-inflammatory cytokine and chemokine production by DCs. It further relates to the method of utilizing LZ-8 as adjuvant to facilitate T cell proliferation and T helper type 1 differentiation induced by DCs.

### DESCRIPTION OF PRIOR ART

Dendritic cells (DCs) are professional antigen-presenting cells and work as a bridge to link innate and adaptive immunity (Steinman, R. M. Nat Med 2007; 13:1155-1159.). DCs are heterogenous but all subsets have intrinsic and cooperative immunoregulatory function (Wu, L. et al, Immunity 2007; 26:741-750; Iwasaki, A. Ann Rev Immunol 2007; 25:381-418.). They can guide the naive T cells into either immunogenic or immune-tolerant direction when exposed to a particular antigen or environment (Abbas, A. K. et al., Nat Immunol 2005; 6:227-228.). When stimulated by inflammatory mediators or microbial pathogens, DCs become mature during their migration to the lymphoid, which dramatically enhances the ability of DCs to activate antigen-specific T cells (Reis e Sousa, C. Nat Rev Immunol 2006; 6:476-483.).

Toll-like receptors (TLRS) play a major role in the innate recognition of pathogen-associated molecular patterns (PAMPs) and initiation of immune responses in DCs (Lee, M. S. et al, Ann Rev Biochem 2007; 76:13.1-13.34.). Upon TLR stimulation, the expression of MHC class II and co-stimulatory molecules, in particular CD40, CD80, and CD86, are upregulated in mature DCs. All these molecules are important for priming T cells (Kawai, T. et al, Semin Immunol 2007; 19:24-32.). According to the key regulatory role of DCs in immune responses, DCs are being developed for the treatment of cancer, allergies and viral infections, and as adjuvants for potent new vaccines to prevent or treat cancer and infectious diseases (Banchereau, J. et al, Nat Rev Immunol 2005; 5:296-306.; Steinman, R. M. et al, Nature 2007; 449:419-426.). Substances that promote DC activation can potentially be applied to immunotherapy and vaccination.

A lot of biologically active natural products have been isolated from Aphyllophorales, many of which are known as polypores. Polypores are a large group of terrestrial fungi of the phylum Basdiomycota (basidiomycetes), and they along with certain Ascomycota are a major source of pharmacologically active substances (Zjawiony, J. K. et al, J Nat Prod 2004; 67:300-310.). Ganoderma lucidum (Leyss. ex Fr.) Karst. (Ling-Zhi or Reishi), a well-known medicinal fungus, is a lamella-less basidiomycetous fungus assigned to the family Polyporaceae. The medicinal properties of this mushroom, including many health-promoting and therapeutic effects, have been recognized for many centuries in Asia. Evidence has been accumulated concerning the medical application of G. lucidum in the treatment of various diseases, such as tumor, cancer metastasis, hypertension, hepatitis, gastritis, arthritis, bronchitis, asthma, anorexia and immunological disorders (Lin, Z., Acta Pharmacol Sin 2004; 25:1387-1395; Lin, Z. J Pharmacol Sci 2005; 99:144-153.). Recently, researchers have studied the biological effect of bioactive components extracted or purified from fruiting bodies, pure culture mycelia, and culture filtrate (cultured broth) (Shiao, M. S., The Chem Record 2003; 3:172-180.). However, the medical application of G. lucidum still remains to be supported by more convincing evidences.

Pharmaceutically active compounds isolated from basidiocarps and mycelium of G. lucidum include polysaccharides, triterpenoids, proteins, lectins, sterols, alkaloids, nucleotides, lactones and fatty acids (Zhou, X. et al, Phytochemistry 2006; 67:1985-2001.). Many studies have indicated that polysaccharides are major effective components of G. lucidum (Hsu, H. Y. et al, J Immunol 2004; 173:5989-5999.). In addition, some investigations have been carried out that the natural triterpenoids in G. lucidum have anti-inflammation, anti-hepatitis, and anti-cancer activities (Wang, G et al, Int Immunopharmacol 2007; 7:864-870.). In contrast to polysaccharides and triterpenoids, not many studies emphasized the function of proteins in G. lucidum research (Jeurink, P. V et al, Int Immunopharmacol 2008; 8:1124-1133.). An immunomodulatory protein with a molecular mass of 12.4 kDa, designated LZ-8, has been isolated from G. lucidum mycelia (Kino, K. et al, J Biol Chem 1989; 264:472-478.). It has also been isolated from another specis G. tsugae and named FIP-gts (Lin, W. H. et al, J Biol Chem 1997; 272:20044-20048.). Some studies have shown that the immunomodulatory effect of LZ-8 on autoimmunity and transplantation, and LZ-8 works as a mitogen to activate T cells (Hsu, H. Y. et al., J Cell Physiol 2008; 215:15-26.). Because DCs play a central role in immune system, the effect of G. lucidum on DCs had been studied but only polysaccharides had been identified (Lin, Y. L. et al, Mol Pharmacol 2006; 70:637-644.). Thus, there is no report to reveal the effect of LZ-8 protein on DCs.

### BRIEF DESCRIPTION OF THE DRAWINGS

FIG. 1 shows the production of cytokines and chemokines by BMDCs in response to LZ-8 stimulation. (A) Dose response curve. BMDCs were incubated with LZ-8 at indicated doses for 6 hours in the presence of Brefeldin A for the last 4 hours. The percentages of TNFα-producing CD11c⁺ cells were determined by flow cytometry. (B and C) BMDCs were incubated with LZ-8 at indicated doses for 24 hours (6 hours for TNFα). Supernatants were collected and (B) TNFα, IL-1α, IL-1β, IL-2, IL-6, and IL-12 (C) MCP-1, MIP-1α, MIP-1β, and RANTES were determined by ELISA. Error bars indicate±SD of triplicate samples. All data are representative of three independent experiments.

FIG. 2 shows that BMDC activation was induced by LZ-8 protein but not contamination. BMDCs were incubated with LPS (20 ng/ml) or LZ-8 (5 μg/ml) for 6 hours in the presence of Brefeldin A for the last 4 hours. The percentages of TNFα-producing CD11c⁺ cells were determined by flow cytometry and shown above the regional marker. (A) LPS, LZ-8, and the background control were tested. (B) LPS and LZ-8 were treated with polymyxin B (5 μg/ml, Sigma-Aldrich) for 30 min before adding to BMDCs. (C) LZ-8 and LPS were incubated with proteinase K for 1 hour before treatment. (D) LZ-8 and LPS were boiled for 25 and 50 min before treatment. Data are representative of three independent experiments.

FIG. 3 shows the promotion of BMDC maturation by LZ-8. (A) BMDCs were incubated with LZ-8 (5 μg/ml) (black line) or were untreated (dark gray line) for 16 hours. Light gray line represents staining with an isotype-matched control antibody. DC maturation was determined by flow cytometry. Cells were stained with mAbs specific for I-A^{b}, CD86, CD80, CD40, CD54 and CD119. (B) BMDCs were incubated with LZ-8 (5 μg/ml), control (background of LZ-8 solution), and LPS (20 ng/ml), or were untreated for 16 hours. Endocytosis of DCs was determined by the uptake of Dextran-FITC at 4° C. (background, gray line) or 37° C. (black line). The percentages of Dextran-FITC⁺ cells are shown above the regional marker. The data shown are gated on CD11c⁺ cells. All data are representative of three independent experiments.

FIG. 4 shows the activation of T cell by LZ-8-treated BMDCs. (A) CD8⁺/CD4⁺ T cells were isolated from OT-I/OT-II mice and co-cultured with LZ-8 (5 μg/ml)- and LPS (20 ng/ml)-activated BMDCs in the presence of OVA₂₅₇₋₂₆₄/OVA₃₂₃₋₃₃₉ peptide (1 μg/ml) at DC:T cell=1:2 for 72 hours. T cell proliferation was determined by [³H]thymidine incorporation in left panels. IFN-γ production was measured by ELISA in right panels. (B) C57BL/6 mice were immunized with OVA₃₂₃₋₃₃₉ peptide (10 μg) mixed with IFA only or IFA+LZ-8 (10 μg) via footpad injection. Draining lymph node cells were collected after 10 days and cultured in 96-well plates with indicated concentration of OVA₃₂₃₋₃₃₉ peptide for 3 days. T cell proliferation was determined by [³H]thymidine incorporation. Error bars indicate±SD of triplicate samples. All data are representative of three independent experiments.

FIG. 5 shows the activation of MAPKs and NF-κB induced by LZ-8 stimulation in BMDCs. BMDCs were harvested, starved, treated with LZ-8 (10 μg/ml), and then lysed at indicated time points. Samples were separated on SDS-PAGE gels, transferred to nitrocellulose membrane, and then analyzed by western blotting. The JNK, ERK, and p38 MAPK proteins with and without phosphorylation were detected by anti-phosphospecific and anti-protein Abs, respectively. IκB degradation was determined by anti-IκB Ab. Data shown are representative of three independent experiments.

FIG. 6 shows the activation of macrophages by LZ-8. RAW264.7 cells were incubated with LZ-8 (5 μg/ml) and LPS (100 ng/ml) for 16 hours. Supernatants were collected and TNFα production was determined by ELISA. Data shown are representative of two independent experiments.

FIG. 7 shows activation of human monocyte-derived DCs (MoDCs) by LZ-8. Immature MoDCs were treated with LPS (1 ug/ml), poly(I:C) (pIC, 25 ug/ml) or various concentrations of LZ-8 for 48 hours. (A) The cells were stained with Abs for CD80, CD86 (Immunotech), and CD83 (BD PharMingen), and then analyzed by flow cytometry. Data are representative of three independent experiments. (B) The culture supernatant was collected and analyzed for cytokine production by human cytometric bead array kits. Error bars indicate SD of three independent experiments.

### SUMMARY OF THE INVENTION

The present invention provides a method for enhancing innate and adaptive immunity by activating dendritic cells (DCs) and macrophages, comprising administering a subject LZ-8 protein. The present invention also provides a method for enhancing the immunogenicity of an antigen, comprising administering a subject with LZ-8 protein-fused antigen.

### DETAILED DESCRIPTION OF THE INVENTION

DCs play a central role in the initiation and regulation of immune response, and serve as a bridge to link innate and adaptive immunity. Matured DCs can attract, interact and activate naive T cells to initiate primary immune response. DCs are also able to directly activated NK cells and can produce large amount of interferons upon encounter with viral pathogens. The present invention provides a method for enhancing innate and adaptive immunity by activating DCs and macrophages. The feature of the method is administering LZ-8 protein. By administering LZ-8 protein, the DCs are activated and produce cytokines and chemokines.

Previous studies have identified the immunomodulatory activity of LZ-8. However, whether LZ-8 protein exerts any effect on DCs is still unclear. The present invention discovers that LZ-8 protein can activate DCs and macrophage. Therefore, innate and adaptive immunity are enhanced by administering the LZ-8 protein present herein the present invention.

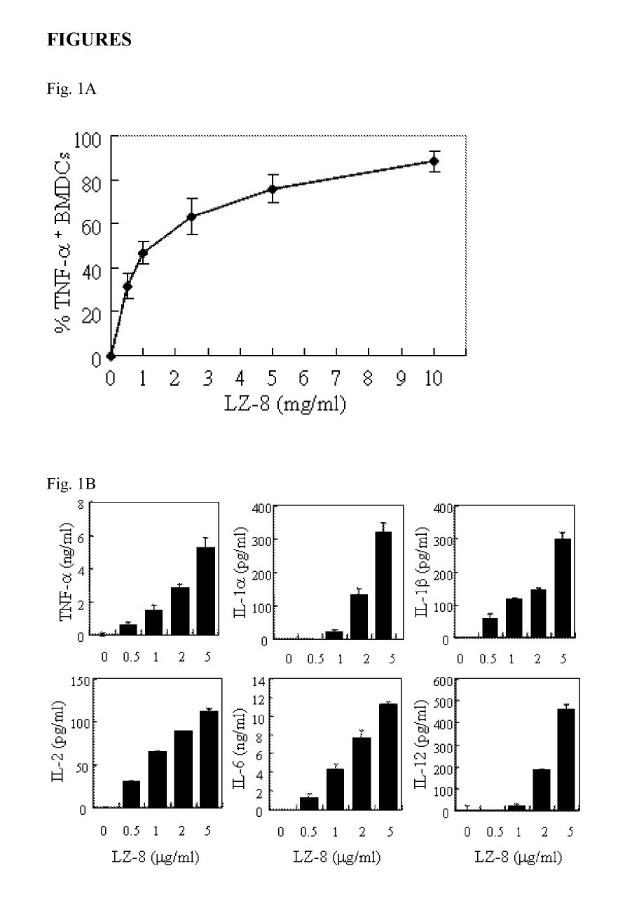

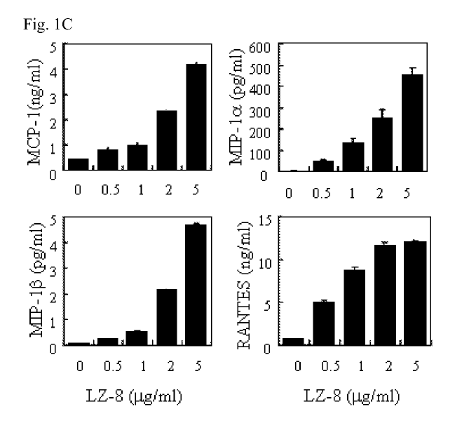

The LZ-8 protein regulates adaptive immunity through promoting DCs activation and maturation. Since the pro-inflammatory cytokine production is a major evidence for DC activation, the induction of TNF by LZ-8 in bone marrow-derived DC (BMDCs) is examined. BMDCs generated TNF after LZ-8 treatment in a dose-dependent manner (FIG. 1A), indicating that LZ-8 can potentially activate DCs. In addition to TNF, intracellular IL-6 and IL-12 p40 are also detectable. The other cytokines are also measured after LZ-8 treatment. As shown in FIG. 1B, LZ-8-treated BMDCs secrete TNF α, IL-1α, IL-1β, IL-2, IL-6, and IL-12 significantly. The chemokines production, e.g. monocyte chemoattractant protein 1 (MCP-1), macrophage inflammatory protein 1α (MIP-1α), macrophage inflammatory protein-1β (MIP-1β), and regulated upon activation, normal T-cell expressed and secreted (RANTES), are also produced by LZ-8-stimulated BMDCs (FIG. 1C). The LZ-8 protein in the present invention activates DCs to secrete cytokines and chemokines and enhances the adaptive immune response.

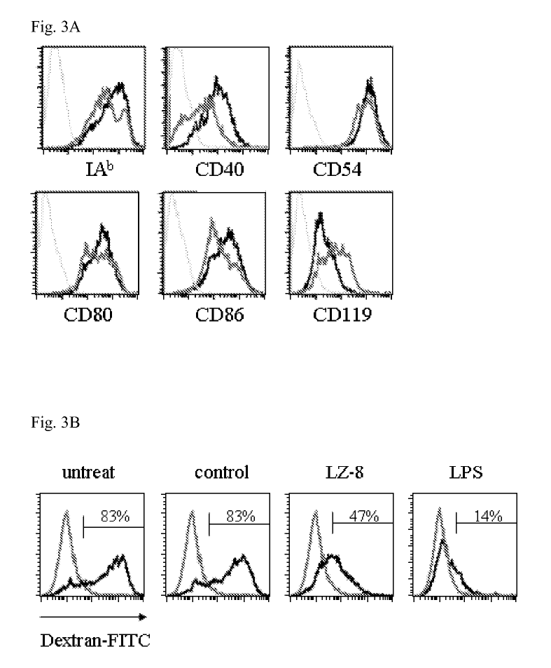

The maturation is a key step for the regulation function of DCs. The maturation states of BMDCs after LZ-8 stimulation are examined. LZ-8 augmented BMDC maturation by upregulating the expression of MHC class II, CD40, CD54 (ICAM-1), CD80, CD86 and downregulating CD119 (IFN-γ receptor) expression (FIG. 3A). DC activation is also accompanied by reduced endocytosis of large molecules. The LZ-8 treatment reduces uptake of FITC-labeled dextran by BMDCs (FIG. 3B). These results demonstrate that LZ-8 in the present invention can activate DCs and promote DC maturation.

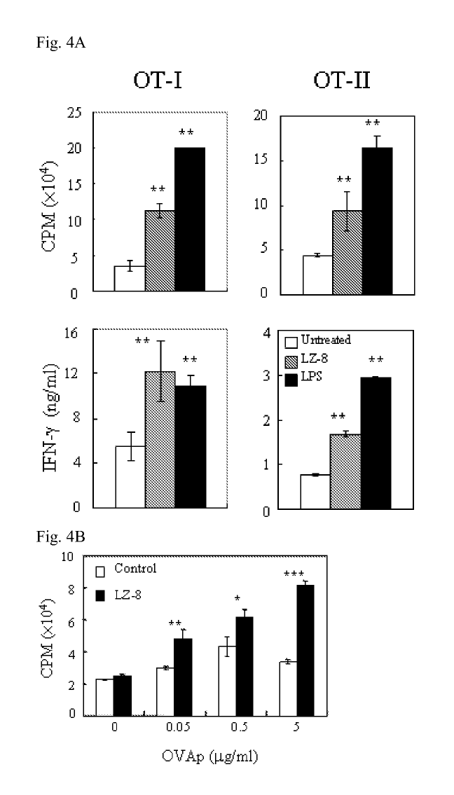

The present invention demonstrates that LZ-8-stimulated BMDCs induce Ag-specific T cell activation both in vitro and in vivo, and also support the results that LZ-8 causes a relative enhancement in DC maturation. Induction of antigen-specific T cell activation is the primary function of mature DCs. LZ-8-activated BMDCs promoted T cell proliferation (FIG. 4A, upper panels). The total T cells activated by LZ-8-stimulated BMDCs produce more IFN-γ (FIG. 4A, lower panels). The present invention also shows that the proliferative activity of T cells isolated from LZ-8-immunized mice is elevated by the LZ-8 treatment in a subunit vaccine model (FIG. 4B). Therefore, based on the discovery, the present invention further provides a method for enhancing the immunogenicity of an antigen, comprising administering a subject with LZ-8 protein-fused antigen. The LZ-8 protein used herein is as adjuvant to enhance the immune response of the subject. In a preferred embodiment, the administration of LZ-8 protein-fused antigen is via injection.

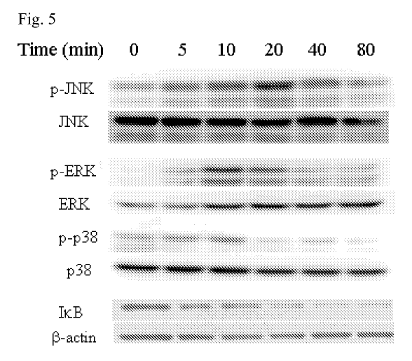

For understanding of biological mechanism, the present invention also provides the pathways involve in the activation of BMDCs by LZ-8. The mitogen-activated protein kinase (MAPKs) signaling pathway and NF-κB pathway are demonstrated involving the activation and maturation of DCs. LZ-8 protein induces the activation of MAPKs (FIG. 5). The MAPKs signaling pathway includes extracellular signal-regulated kinases (ERK), JUN N-terminal kinases (JNK), and stress-activated protein kinase 2A (p38).

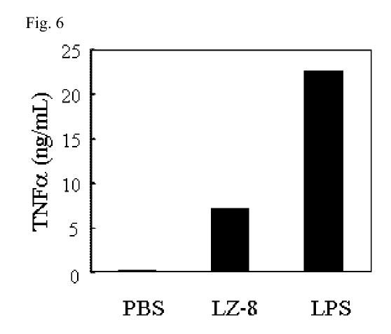

The method of the present invention is not only enhancing innate and adaptive immunity by promoting the activation and maturation of DCs but also enhancing innate immunity by inducing the activation of machrophages. The macrophage RAW264.7 (RAW) cells are incubated with LZ-8 and LPS and the TNFα production is determined by ELISA. As shown in FIG. 6, LZ-8 promoted TNF secretion in RAW cells. The result indicates that LZ-8 can activate macrophages and enhance innate immunity.

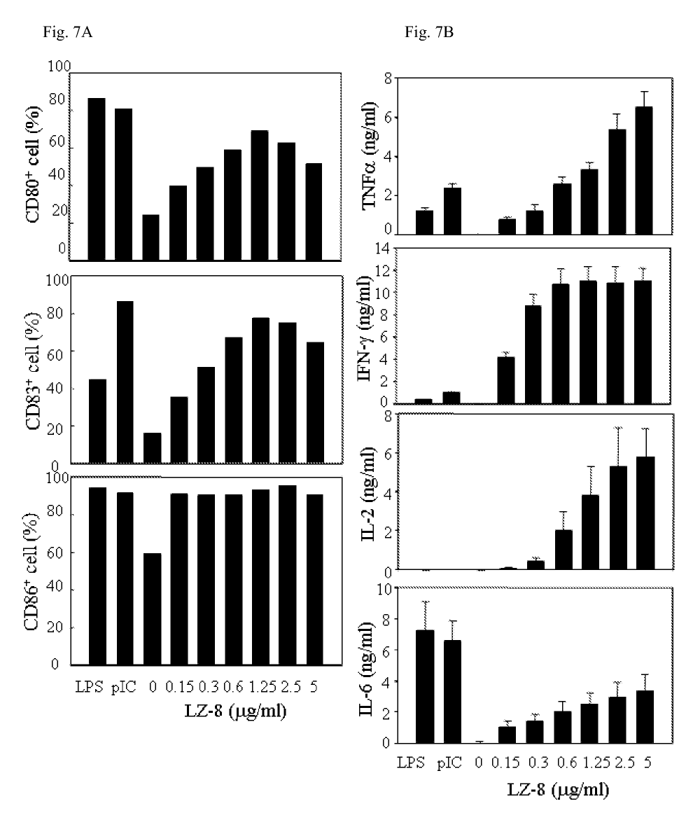

The present invention has demonstrated the effect of LZ-8 on DCs in mouse. Furthermore, since human DCs show some different features from mouse DCs, human monocyte-derived DCs (MoDCs) are also treated with LZ-8 and the cells aggregation are observed. The present invention illustrated that LZ-8 enhances the expression of CD80, CD83, and CD86 in LZ-8-treated MoDCs (FIG. 7A). The production of cytokines is induced by LZ-8 stimulation in the MoDCs (FIG. 7B). The cytokines comprise TNFα, IFN-γ, IL-2, and IL-6. The data demonstrates that LZ-8 can activate human DCs in consistent to its effect on mouse DCs. Therefore, the present invention also provides potential application of LZ-8 in DC-based immunotherapy in a mammal such as human.

The LZ-8 protein in the present invention is isolated from Ganoderma lucidum or prepared by recombinant protein technology in host cells. The host cells can be yeast or bacterium system. The said host cells are Saccharomyces cerevisiae, Pichia pastoris, Hansenula polymorpha, Candida utilis, Candida boidinii, Candida maltosa, Kluyveromyces lactis, Yarrowia lipolytica, Schwanniomyces occidentalis, Schizosaccaromyces pombe, Torulopsis, Arxula adeninivorans, or Aspergillus (A. nidulans, A. niger, A. awamori, A. oryzae), Tricoderma (T. reesei).

The LZ-8 in the present invention also activates human MoDCs in vitro. The present invention provides a method for promoting DCs maturation and function in vitro.

The incidence of some cancers is increased in immunodeficient patients and is increased with age. Based on this concept and discovery, enhancing the immunity of subject can help for treating cancer and preventing the occurrence of cancer. Therefore, the present invention can further use for cancer therapy by promoting the activation and maturation of DCs. The present invention also can be used in DC-based vaccine for cancer therapy and infectious diseases. Therefore, the present invention provides a method further combines with DC-based vaccine.

The present invention also provides a composition, comprising LZ-8 protein-treated DCs. The composition promote the activation and maturation of dendritic cells (DCs) by activating dendritic cells (DCs),

### EXAMPLES

The examples below are non-limiting and are merely representative of various aspects and features of the present invention.

### Materials and Methods

### Mice and DC Cultures

Mouse DCs were prepared from bone marrow isolated from C57BL/6 mice (National Laboratory Animal Center, Taipei, Taiwan) as previously described (Chu, C. L., and C. A. Lowell, J Immunol 2005; 175:2880-2889.). OT-I and OT-II TCR transgenic mice were provided by Dr. Clifford Lowell (UCSF, CA). All mice were housed in the barrier facility at NHRI (Taiwan) under an Institutional Animal Care and Use Committee-approved protocol.

### Preparation of Recombinant LZ-8

LZ-8 from G. lucidum was cloned and expressed in Saccharomyces cerevisiae. (S. cerevisiae). Cells expressing LZ-8 (or empty vector as control) were then spun down and passed through a cell disrupter. The lysate was centrifuged and the supernatant was passed through a 0.2 μm vacuum filter and molecular sieves to obtain proteins between 10 kDa and 100 kDa. The filtrate was further purified using FPLC with Superdex 75 columns (GE Healthcare). The purity was determined to be 98% through FPLC.

### Preparation of Human MoDCs

MoDCs were generated from peripheral blood mononuclear cells (PBMCs) as previously described (Chu, C. L. et al. E J Immunol 2008; 38:166-173.). Briefly, PBMCs were enriched by density gradient centrifugation with Ficoll-Paque and incubated for 2 hours at 37° C. in AIM-V medium (Invitrogen), and then the adherent cells were cultured in X-VIVO15 medium containing 2% heat-inactivated autologous plasma, 1000 U/mL human IL-4 (Strathmann Biotec AG), and 500 U/mL granulocyte-macrophage-colony-stimulating factor (Leukomax; Novartis International AG, Basel, Switzerland). On Day 7, loosely attached or floating cells were collected as immature DCs.

### Statistical Analysis

Significances of cytokine and chemokine production and T cell proliferation of LZ-8 treatment in comparison with control were determined using a Student's t-test with 2 sample equal variance with a 2 tailed distribution. Error bars indicated mean±SD of triplicate samples. The p value <0.05 is considered to be significant.

### Example 1

### LZ-8 Stimulated BMDCs to Produce Cytokines and Chemokines

To determine the effect of LZ-8, induction of TNFα was examined in BMDCs. BMDCs generated TNFα after LZ-8 treatment in a dose-dependent manner (FIG. 1A), indicating that LZ-8 can potentially activate DCs. In addition to TNFα, intracellular IL-6 and IL-12 p40 were also detectable (data not shown). Quantitatively, we used ELISA to measure the cytokines secreted by LZ-8-treated BMDCs. As shown in FIG. 1B, LZ-8-treated BMDCs secreted TNFα, IL-1α, IL-1β, IL-2, IL-6, and IL-12 significantly. We also determined the chemokine production and MCP-1, MIP-1α, MIP-1β, and RANTES were generated by LZ-8-stimulated BMDCs (FIG. 1C).

### Example 2

### BMDCs were Activated by LZ-8 Protein but not Contamination

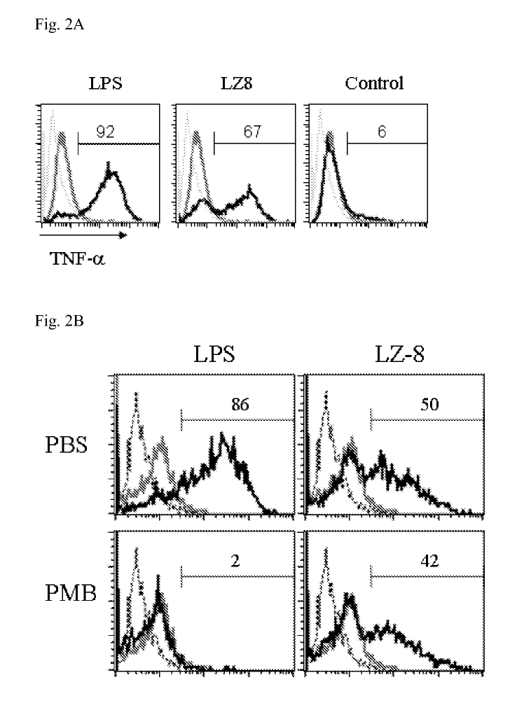

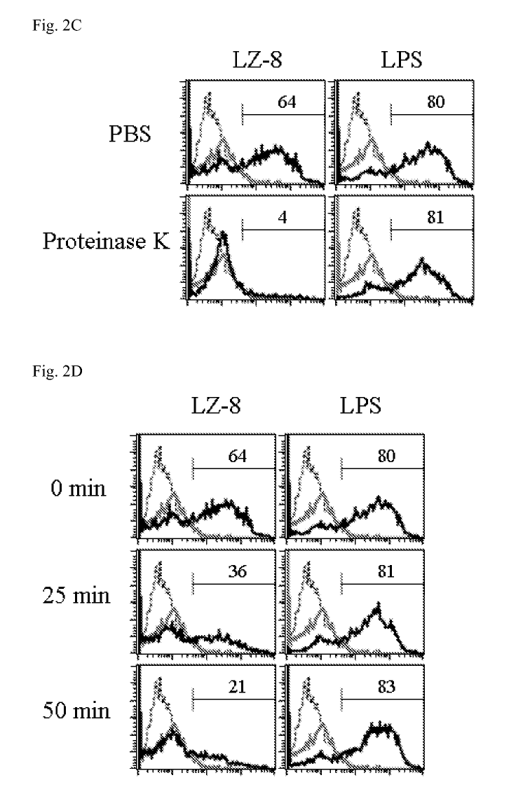

The LZ-8 protein used herein was from yeast-expressed recombinant protein. To exclude the possibility of contamination of yeast components during LZ-8 preparation, the background solution prepared from yeast expressing empty vector was used as a control. As shown in FIG. 2A, the control solution had no effect on BMDCs for TNFα production, excluding the contamination effect. In addition, Polymyxin B did not significantly inhibit the activity of LZ-8, indicating that the DC activation did not due to endotoxin contamination (FIG. 2B).

Furthermore, the LZ-8 protein was inactivated before treating BMDCs by proteinase K at 37° C. for 1 hour or heated for 25 and 50 min at 100° C. As showed in FIGS. 2C and 2 D, the proteinase K-digested and heat-inactivated LZ-8 lost the ability to stimulate BMDCs, suggesting the activity comes from LZ-8 protein itself. These data demonstrated that the stimulating activity of LZ-8 on DCs was not cause by contamination of yeast components.

### Example 3

### LZ-8 Promoted BMDC Maturation

The maturation state of BMDCs after LZ-8 stimulation was examined by flow cytometry. Six-day-cultured BMDCs were treated with LZ-8 (5 μg/ml) for 16 hours. Then, the cells were blocked with anti-CD16/CD32 mAb 2.4G2 (BD Pharmingen), stained with mAbs against CD11c, CD40, CD54, CD80, CD86, CD119, and I-A^{b} (Biolegend), and analyzed by flow cytometry. As shown in FIG. 3A, the LZ-8 upregulated the expression of MHC class II, CD40, CD54 (ICAM-1), CD80, CD86 and downregulated CD119 (IFN-γ receptor) expression.

In addition, it is known that DC activation is accompanied by reduced endocytosis of large molecules. Therefore, endocytosis of BMDCs was also examined. The untreated or treated BMDCs were incubated with 200 μg/ml Dextran-FITC (M.W. ˜77 kD, Sigma-Aldrich) for 1 hour at 4° C. or 37° C. Cells were washed with cold PBS, stained with anti-CD11c mAb, and then analyzed by flow cytometry. As shown in FIG. 3B, the LZ-8 treatment reduced uptake of FITC-labeled dextran by BMDCs. Consistently, the control solution did not change the endocytosis of BMDCs. These results demonstrated that LZ-8 can activate DCs and promote DC maturation.

### Example 4

### LZ-8-Treated DCs Induced T Cell Activation

Induction of antigen-specific T cell activation is the primary function of mature DCs. T cells isolated from OT-I or OT-II TCR transgenic mice and cells were co-cultured with LZ-8-treated, OVA₂₅₇₋₂₆₄ (OVA_{P1})- or OVA₂₅₇₋₂₆₄ (OVA_{P2})-pulsed BMDCs for 72 hours. Then, the proliferation of T cells was determined by [³H]thymidine incorporation. The procedure was as follow. Antigen presentation by BMDCs was determined as described previously. Briefly, BMDCs were purified by using an EasySep Positive Selection Kit (StemCell Technology), seeded in 96-well flat-bottom plates (Costar Corning) and adding 1 μg/ml OVA_{P1} or OVA_{P2} with or without LZ-8, and incubated for 3 hours. T cells were isolated from OT-I or OT-II TCR transgenic mice with EasySep Positive Selection Kit and added to DC cultures at DC/T cell=½. Cells were incubated for 72 hours and T cell proliferation was determined by [³H]thymidine incorporation. As shown in FIG. 4A (upper panels), LZ-8-activated BMDCs promoted more T cell proliferation than control cells in vitro. In addition, total T cells activated by LZ-8-stimulated BMDCs also produced more IFN-γ than that by control cells (FIG. 4A, lower panels).

Further examination of T cells activation was performed in vivo. In order to determine the induction of T cell activation by LZ-8 in vivo, a subunit vaccine model was performed to evaluate the effect of LZ-8 on T cell priming. For recall assays, C57BL/6 mice were immunized with 10 μg OVA_{P2} mixed with incomplete Freund's Adjuvant (IFA, Sigma-Aldrich) only or IFA+LZ-8 (10 μg) via footpad injection. Draining lymph node cells were isolated from immunized mice after 10 days and cultured with OVA_{P2} for 3 days. T cell proliferation was determined by [³H] thymidine incorporation. The results showed that cells isolated from LZ-8-immunized mice showed more proliferation than cells from control mice in response to OVAp₂ along (FIG. 4B). These data revealed that LZ-8-stimulated BMDCs induce Ag-specific T cell activation both in vitro and in vivo, and also support the conclusion that LZ-8 causes a relative enhancement in DC maturation.

### Example 5

### Activation of MAPKs and NF-kB Induced by LZ-8 Stimulation in BMDCs

In order to explored the molecular mechanisms underlying cell activation by LZ-8, the activations of MAPKs and NF-κB in LZ-8-treated BMDCs were examined. BMDCs were treated with LZ-8 and both the inactive and active forms of JNK, ERK, and p38 MAPK were analyzed by Western blotting. The procedures were as follow. BMDCs were harvested, starved for 3 hours, and then treated with LZ-8 (10 μg/ml). Cells were lysed, boiled, separated on SDS-PAGE gels, and then transferred to Immunobilon NC membrane (Millipore). After blocking, blots were incubated with anti-phosphospecific Abs against p38 MAPK (Thr180/Tyr182), ERK (Thr202/Tyr204), and JNK (Thr183/Tyr185) (Cell Signaling Technology) or anti-p38 MAPK (Millipore), ERK (BD Transduction Laboratories), JNK, and IκB (Santa Cruz Biotechnology) Abs, followed by HRP-conjugated secondary Abs (Chemicon). Immunoreactivity was detected using the ECL detection reagent (Pierce). The results showed that LZ-8 induced activation of these MAPKs (FIG. 5). In addition, LZ-8 also induced degradation of IκB, suggesting that this protein can activate the NF-κB pathway. These results suggested that LZ-8 promotes DC maturation and function by activating MAPKs and NF-κB pathways.

### Example 6

### Activation of Macrophages by LZ-8

The RAW264.7 cell line was cultured in RPMI containing 10% fetal bovine serum (FBS), 2 mM glutamine, 1% nonessential amino acid, and 1 mM sodium pyruvate. Cells were maintained in a humidified incubator at 37° C. in 5% CO₂. For activation, the cells were treated with LZ-8 (5 mg/ml) or LPS (100 ng/ml) for 16 hours, and the supernatants were collected. TNFa production was measured by ELISA. As shown in FIG. 6, LZ-8 promoted TNFα secretion in RAW cells. These results indicated that LZ-8 can activate macrophages and enhance innate immunity.

### Example 7

### Activation of Human MoDCs by LZ-8

To explore the effect of LZ-8 on human DCs, human monocyte-derived DCs (MoDCs) treated with LZ-8 showed cell aggregation in culture (data not shown). Then, the maturation state of LZ-8-treated MoDCs was examined by flow cytometry. The cells were stained with Abs for CD80, CD86 (Immunotech), and CD83 (BD PharMingen), and then analyzed by flow cytometry. As shown in FIG. 7A, LZ-8 induced the expression of CD80, CD83, and CD86 in MoDCs. These results were in agreement with that in mouse BMDCs.

In addition, the cytokine production by MoDCs after LZ-8 stimulation was also examined. For detecting the cytokines production, supernatants were collected from MoDC cultures with TLR ligands or LZ-8 as indicated after 24 hours. The cytokines were measured by using a human cytometric bead array kit (BD PharMingen). As shown in FIG. 7B, TNFα, IFN-γ, IL-2, and IL-6 were detected. Surprisingly, LZ-8 dramatically induced the production of IFN-γ and IL-2 by MoDCs when compared to TLR-activated cells. These data demonstrated that LZ-8 can activate human DCs in consistent to its effect on mouse DCs.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for enhancing innate and adaptive immunity by activating dendritic cells (DCs) and macrophages, comprising administering a subject LZ-8 protein or LZ-8-treated dendritic cells (DCs).

2. The method of claim 1, wherein the LZ-8 protein is isolated from Ganoderma lucidum or prepared by recombinant protein technology in yeast or bacterium system.

3. The method of claim 1, wherein the activation dendritic cells (DCs) produces cytokines or chemokines.

4. The method of claim 1, wherein the activation dendritic cells (DCs) further comprises a maturation of the dendritic cells (DCs).

5. The method of claim 1, wherein the macrophages produce cytokines.

6. The method of claim 4, wherein the maturation of dendritic cells (DCs) is through the activation of mitogen-activated protein kinase (MAPK) pathway or NF-κB.

7. The method of claim 4, wherein the maturation of dendritic cells (DCs) induces T cells activation and proliferation.

8. The method of claim 3, wherein the cytokines are selected from the group consisting of TNFα, IL-1beta (IL-1β), interleukin-6 (IL-6), interleukin-10 (IL-10) and interleukin-12 (IL-12).

9. The method of claim 3, wherein the chemokines are selected from the group consisting of monocyte chemoattractant protein 1 (MCP-1), macrophage inflammatory protein 1α (MIP-1α), macrophage inflammatory protein-1β (MIP-1β), and regulated upon activation, normal T-cell expressed and secreted (RANTES).

10. The method of claim 6, wherein the mitogen-activated protein kinase (MAPK) pathway are selected from the group consisting of JNK, ERK and p38.

11. The method of claim 7, wherein the T cells produce cytokines selected from the group consisting of interleukin-2 (IL-2), interleukin-4 (IL-4) and interferon gamma (IFN-γ).

12. A method for enhancing immunogenicity of an antigen, comprising administering a subject with LZ-8 protein-fused antigen.

13. The method of claim 12, wherein the LZ-8 protein-fused antigen is prepared by recombinant protein technology in yeast or bacterium system.

14. The method of claim 12, wherein the LZ-8 protein-fused antigen is used as adjuvant in vaccine.

15. The method of claim 14, wherein the immunogenicity of an antigen is enhance by T cell activation induced by LZ-8 in vivo.

16. The method of claim 1, further combines with DC-based vaccine.

17. A composition which comprises LZ-8 protein treated DCs.
